(19) United States
(12) Patent Application Publication
(10) Pub. No.: US 2024/0120109 A1
(43) Pub. Date: Apr. 11, 2024

(54) ARTIFICIAL INTELLIGENCE ARCHITECTURE FOR PROVIDING LONGITUDINAL HEALTH RECORD PREDICTIONS

(71) Applicant: GenHealth, Inc., Vienna, VA (US)

(72) Inventors: Ritwik Sahu, Vienna, VA (US); Eric Marriott, Princeton, MA (US); Ethan Siegel, Boston, MA (US); Donald Rucker, Arlington, VA (US)

(73) Assignee: GenHealth, Inc., Vienna, VA (US)

(21) Appl. No.: 18/482,409

(22) Filed: Oct. 6, 2023

Related U.S. Application Data
(60) Provisional application No. 63/414,631, filed on Oct. 10, 2022.

(51) Int. Cl. G16H 50/30; G16H 10/60

(52) U.S. Cl. CPC G16H 50/30; G16H 10/60

(57) ABSTRACT

Disclosed embodiments relate to systems and methods for providing medical data to predict patient events. Techniques include accessing, from one or more data sources, patient data associated with a patient; extracting, from the patient data, a sequence of events associated with the patient; inputting the sequence of events associated with the patient into a trained machine learning model; receiving, as an output of the trained machine learning model, a sequence of predicted events associated with the patient, the sequence of predicted events including at least one event that is predicted to occur; and transmitting an indication of the at least one predicted event.

### CROSS-REFERENCE TO RELATED APPLICATION

This application claims the benefit of priority of U.S. Provisional Application No. 63/414,631, filed Oct. 10, 2022. The foregoing application is incorporated herein by reference in its entirety.

### BACKGROUND

### Technical Field

The present disclosure relates generally to artificial intelligence, and more specifically, to techniques for predicting future events associated with patients or other individuals.

### Background Information

The healthcare industry continues to seek reliable and insightful methods to predict costs, treatment plans, and events affecting medical care for patients and other individuals. For example, it may be beneficial to ingest historical data associated with a patient or group of patients and, based on this data, predict future events associated with the patient or group of patients. These predictions may improve the level of care a patient receives, allow for better planning by healthcare providers, and allow for more accurate risk, quality, and cost (among other future care) predictions for patients, health plans, and providers, as well as numerous other benefits.

However, the sheer volume and heterogeneity of different sources of patient data and the complexity of understanding and analyzing this data are significant barriers for developing reliable prediction tools. For example, healthcare providers interested in predicting future patient events may look to electronic health records (EHRs) of patients to plan treatment and other aspects of patient care. With these massive data sets available for each patient and the large number of patients that may be associated with healthcare organizations, it is often impossible to manually review the entirety of the patient history. Moreover, in many cases, indicators of future events may exist in intricate patterns, markers, or other data in a medical record that may be undetectable by human reviewers. Accordingly, even if there was time to manually review histories for each patient, such a review would likely be ineffective for making predictions.

Some existing techniques employ the use of machine learning algorithms to predict patient events. These techniques, however, often provide limited functionality to predict a specific type of event. Further, these algorithms often are limited to EHRs and do not take into account vast amounts of other forms of data for a patient that may be available. Accordingly, in view of these and other deficiencies in current techniques, technical solutions are needed to efficiently and accurately predict a variety of future events associated with a patient.

### SUMMARY

The disclosed embodiments describe non-transitory computer readable media, systems, and methods for predicting patient events. For example, in an embodiment, a computer-implemented method for providing medical data to predict patient events is disclosed. The method may comprise accessing, from one or more data sources, patient data associated with a patient; extracting, from the patient data, a sequence of events associated with the patient; inputting the sequence of events associated with the patient into a trained machine learning model; receiving, as an output of the trained machine learning model, a sequence of predicted events associated with the patient, the sequence of predicted events including at least one event that is predicted to occur; and transmitting an indication of the at least one predicted event.

In another embodiment, a system for using an expert system for providing medical data to predict patient events may include at least one processor comprising circuitry and a memory. The memory may include instructions that when executed by the circuitry cause the at least one processor to access, from one or more data sources, patient data associated with a patient; extract, from the patient data, a sequence of events associated with the patient; input the sequence of events associated with the patient into a trained machine learning model; receive, as an output of the trained machine learning model, a sequence of predicted events associated with the patient, the sequence of predicted events including at least one event that is predicted to occur; and transmit an indication of the at least one predicted event.

Aspects of the disclosed embodiments may include tangible computer-readable media that store software instructions that, when executed by one or more processors, are configured for and capable of performing and executing one or more of the methods, operations, and the like consistent with the disclosed embodiments. Also, aspects of the disclosed embodiments may be performed by one or more processors that are configured as special-purpose processor(s) based on software instructions that are programmed with logic and instructions that perform, when executed, one or more operations consistent with the disclosed embodiments.

It is to be understood that both the foregoing general description and the following detailed description are exemplary and explanatory only, and are not restrictive of the disclosed embodiments, as claimed.

### BRIEF DESCRIPTION OF THE DRAWINGS

The accompanying drawings, which are incorporated in and constitute a part of this specification, illustrate disclosed embodiments and, together with the description, serve to explain the disclosed embodiments. In the drawings:

FIG. 1 illustrates an example system environment for predicting events associated with a patient or other individual, consistent with the disclosed embodiments.

FIG. 2A is a block diagram showing an example computing device, consistent with the disclosed embodiments.

FIG. 2B is a block diagram showing an example server, consistent with the disclosed embodiments.

FIG. 3 is a block diagram showing example data sources from which patient event data may be derived, consistent with the disclosed embodiments.

FIG. 4 is a block diagram illustrating an example process for training and applying a machine learning model consistent with the disclosed embodiments.

FIG. 5 is a block diagram illustrating an example output sequence that may be generated using trained model, consistent with the disclosed embodiments.

FIG. 6 is a block diagram illustrating an example statistical analysis that may be performed, consistent with the disclosed embodiments.

FIG. 7 is a flowchart showing an example process for providing medical data to predict patient events, consistent with the disclosed embodiments.

### DETAILED DESCRIPTION

In the following detailed description, numerous specific details are set forth in order to provide a thorough understanding of the disclosed example embodiments. However, it will be understood by those skilled in the art that the principles of the example embodiments may be practiced without every specific detail. Well-known methods, procedures, and components have not been described in detail so as not to obscure the principles of the example embodiments. Unless explicitly stated, the example methods and processes described herein are not constrained to a particular order or sequence, or constrained to a particular system configuration. Additionally, some of the described embodiments or elements thereof can occur or be performed simultaneously, at the same point in time, or concurrently.

The techniques for predicting patient events described herein overcome several technological problems relating to artificial intelligence, event prediction, and performance in the fields of healthcare or other industries. As discussed above, the complexity of understanding and analyzing this patient data is a significant barrier for developing reliable prediction tools. In many cases, there may be indicators that an event associated with a patient or other individual is likely to occur, but often the indicators for the event are subtle and distributed across many disparate data sources. Even if all of this data were accessible, the trends in the data that may indicate a future event are often imperceivable to a human observer. Developing tools to detect these indicators is thus equally challenging.

To address these technological challenges, the disclosed techniques implement a machine learning model trained on large sets of sequences of events. Moreover, these events are extracted from a wide variety of different data sources, thus representing a full picture of a patient's history, including many factors not typically represented in a patient's medical data. Based on an input sequence of events, the trained model may be configured to predict events not appearing in the input sequence. For example, this may include events predicted to occur, thus providing valuable insights into the treatment, care, and prognosis of a patient.

In some embodiments, the disclosed techniques may further allow for evaluation of hypothetical events, such as possible treatments for a patient. For example, the input sequence may include events that have not yet occurred but that are hypothetical. The trained model may be used to predict outcomes or future events based on these hypothetical events. The trained model may further be used to determine probabilities of events, timeframes for events, or other predictions, which may be invaluable in the medical industry, as well as various other industries.

Reference will now be made in detail to the disclosed embodiments, examples of which are illustrated in the accompanying drawings.

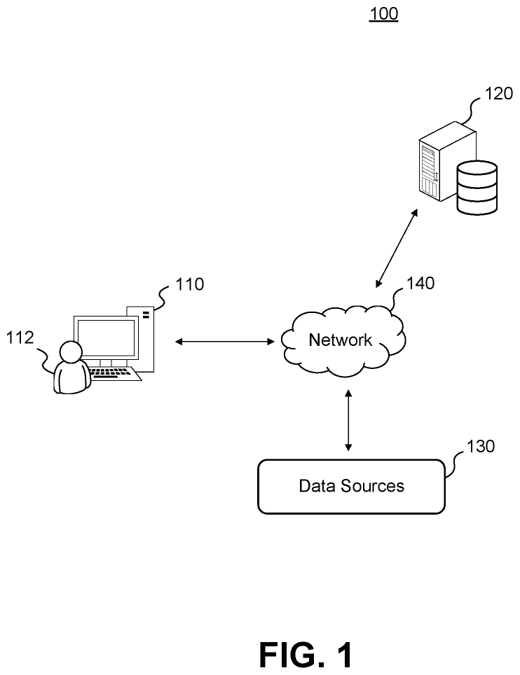

FIG. 1 illustrates an example system environment 100 for predicting events associated with a patient or other individual, consistent with the disclosed embodiments. System environment 100 may include one or more computing devices 110, one or more security servers 120, and one or more data sources 130 as shown in FIG. 1. System environment 100 may represent a system or network environment in which data (e.g., patient data) is accessed from data sources 130 and used to generate predictions of future events associated with a patient, which may allow for improved management of risk, quality, cost, or other aspects of patient care. While the disclosed embodiments are generally described in relation to “patients” it is to be understood that these embodiments may equally apply to a variety of other individuals associated with medical care, such as members of a health plan who have not yet begun treatment, discharged or former patients, or the like.

In some embodiments, system 100 may include a trained artificial intelligence model used to predict various patient events based on patient data. For example, a user 112 may interact with computing device 110 to generate predictions associated with a patient. In some embodiments, this may include causing a request for future events to be transmitted to server 120. Computing device 110 and/or server 120 may access data associated with the patient from data sources 130. The trained artificial intelligence model, which may be executed on computing device 110, server 120, or another component of system 100, may receive patient data (represented as a series of historical patient events) as an input and generate one or more future or predicted patient events.

The various components of system environment 100 may communicate over a network 140. Such communications may take place across various types of networks, such as the Internet, a wired Wide Area Network (WAN), a wired Local Area Network (LAN), a wireless WAN (e.g., WiMAX), a wireless LAN (e.g., IEEE 802.11, etc.), a mesh network, a mobile/cellular network, an enterprise or private data network, a storage area network, a virtual private network using a public network, a nearfield communications technique (e.g., Bluetooth, infrared, etc.), or various other types of network communications. In some embodiments, the communications may take place across two or more of these forms of networks and protocols. While system environment 100 is shown as a network-based environment, it is understood that in some embodiments, one or more aspects of the disclosed systems and methods may also be used in a localized system, with one or more of the components communicating directly with each other.

As noted above, system environment 100 may include one or more computing devices 110. Computing device 110 may include any device that may be used for interacting with patient event data. Accordingly, computing device 110 may include various forms of computer-based devices, such as a workstation or personal computer (e.g., a desktop or laptop computer), a mobile device (e.g., a mobile phone or tablet), a wearable device (e.g., a smart watch, smart jewelry, implantable device, fitness tracker, smart clothing, head-mounted display, etc.), an IoT device (e.g., smart home devices, industrial devices, etc.), or any other device that may be capable of executing instructions associated with patient event data. In some embodiments, computing device 110 may be a virtual machine (e.g., based on AWS™, Azure™, IBM Cloud™, etc.), container instance (e.g., Docker™ container, Java™ container, Windows Server™ container, etc.), or other virtualized instance.

In some embodiments, computing device 110 may be associated with a user 112. User 112 may be any entity that may have or request access to patient event data. For example, user 112 may be a person, an account, an application, a process, an operating system, a service, an organization, or any other entity or attribute associated with one or more components of system environment 100. In some embodiments, user 112 may be a user operating computing device 110 to generate future events associated with a patient. For example, user 112 may be a nurse, a doctor, a practitioner, a patient, a relative, a health advisor, an insurance professional, a medical researcher, or any other individual that may have an interest in predictions for future events associated with a patient. Further, as noted above, the disclosed embodiments are not limited to patient or medical care. Accordingly, user 112 may include a wide variety of other entities interested in predicting future events associated with an individual.

Server 120 may be configured to monitor and/or manage one or more privileges within system environment 100. For example, server 120 may manage one or more privileges associated with user 112 (or computing device 110) required to perform computing operations within system environment 100. In some embodiments, server 120 may represent a privileged access management (PAM) system or other access management system implemented within system environment 100. Alternatively or additionally, server 120 may be a security information and event management (SIEM) resource implemented within system environment 100. Server 120 may be configured to grant, track, monitor, store, revoke, validate, or otherwise manage privileges of various identities within system environment 100. While illustrated as a separate component of system environment 100, it is to be understood that server 120 may be integrated with one or more other components of system environment 100. For example, in some embodiments, server 120 may be implemented as part of target network resource 120, computing device 110, or another device of system environment 100.

In some embodiments, server 120 may be configured to predict one or more patient events, as described in further detail below. For example, server 120 may receive a request by user 112 to generate predictions for patient events (either future events or events that may have already occurred). Server 120 may further receive patient data obtained from data sources 130 and input the patient data into a trained machine learning model. Server 120 may provide an output of the trained model to computing device 110. Alternatively or additionally, computing device 110 may execute the trained machine learning model locally, and may communicate results with server 120.

Data source (or sources) 130 may include a wide variety of resources that may store, generate, or otherwise allow access to data associated with a patient. In some embodiments, data sources 130 may be a single resource, such as a hospital database or other database storing patient data. Alternatively or additionally, data sources 130 may include many discrete data sources, which may have a wide variety of formats. For example, data sources 130 may include a database (or other data storage location), a server, a webpage, a device (e.g., a laptop, a wearable device, an IoT device, etc.), a sensor, a software application, or any other object or entity that may provide access to data. Data sources 130 are described in further detail below, including several nonlimiting examples of data types.

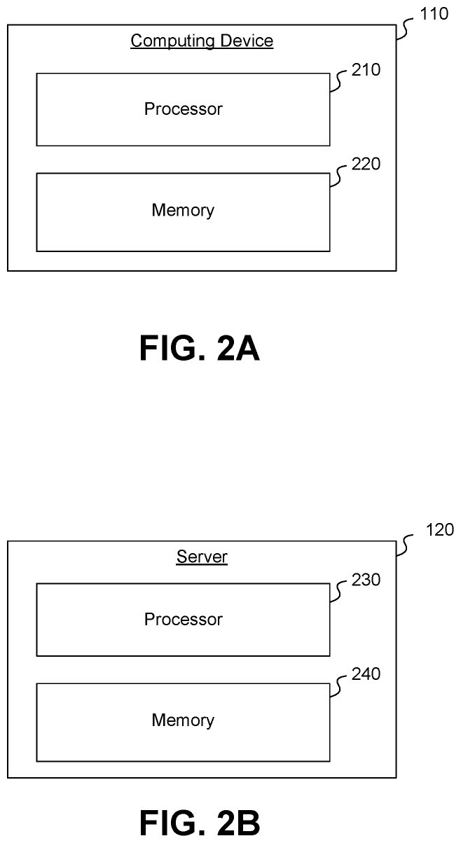

FIG. 2A is a block diagram showing an example computing device 110, consistent with the disclosed embodiments. Computing device 110 may include one or more dedicated processors and/or memories. For example, computing device 110 may include a processor (or multiple processors) 210, and a memory (or multiple memories) 220, as shown in FIG. 2A.

Processor 210 may take the form of, but is not limited to, a microprocessor, embedded processor, or the like, or may be integrated in a system on a chip (SoC). Furthermore, according to some embodiments, processor 210 may be from the family of processors manufactured by Intel®, AMD®, Qualcomm®, Apple®, NVIDIA®, or the like. The processor 210 may also be based on the ARM architecture, a mobile processor, or a graphics processing unit, etc. The disclosed embodiments are not limited to any type of processor configured in server 130. In some embodiments, processor 210 may be a special-use processing device adapted for executing a trained machine learning model.

Memory 220 may include one or more storage devices configured to store instructions used by the processor 210 to perform functions related to computing device 110. The disclosed embodiments are not limited to particular software programs or devices configured to perform dedicated tasks. For example, memory 220 may store a single program, such as a user-level application, that performs the functions associated with the disclosed embodiments, or may comprise multiple software programs. Additionally, processor 210 may, in some embodiments, execute one or more programs (or portions thereof) remotely located from computing device 110. Furthermore, memory 220 may include one or more storage devices configured to store data for use by the programs. Memory 220 may include, but is not limited to a hard drive, a solid state drive, a CD-ROM drive, a peripheral storage device (e.g., an external hard drive, a USB drive, etc.), a network drive, a cloud storage device, or any other storage device.

FIG. 2B is a block diagram showing an example server 120, consistent with the disclosed embodiments. As shown in FIG. 2B, privilege management server 120 may include a processor (or multiple processors) 230, a memory (or multiple memories) 240, and/or one or more input/output (I/O) devices (not shown).

As with processor 210, processor 230 may take the form of, but is not limited to, a microprocessor, embedded processor, or the like, or may be integrated in a system on a chip (SoC). Furthermore, according to some embodiments, processor 230 may be from the family of processors manufactured by Intel®, AMD®, Qualcomm®, Apple®, NVIDIA®, or the like. Processor 230 may also be based on the ARM architecture, a mobile processor, or a graphics processing unit, etc. In some embodiments, processor 210 may be a special-use processing device adapted for executing a trained machine learning model. The disclosed embodiments are not limited to any type of processor configured in server 120.

Further, similar to memory 220, memory 240 may include one or more storage devices configured to store instructions used by the processor 230 to perform functions related to server 120. The disclosed embodiments are not limited to particular software programs or devices configured to perform dedicated tasks. For example, memory 240 may store a single program, such as a user-level application (e.g., a browser), that performs the functions associated with the disclosed embodiments, or may comprise multiple software programs. Additionally, processor 230 may, in some embodiments, execute one or more programs (or portions thereof) remotely located from server 120. Furthermore, memory 240 may include one or more storage devices configured to store data for use by the programs. Memory 240 may include, but is not limited to a hard drive, a solid state drive, a CD-ROM drive, a peripheral storage device (e.g., an external hard drive, a USB drive, etc.), a network drive, a cloud storage device, or any other storage device.

As indicated above, computing device 110 and/or server 120 may access various data associated with one or more patients. Consistent with some embodiments of the present disclosure the data may represent various patient events. As used herein, a “patient event” may include any form of event associated with a patient. In some embodiments, a patient event may be a medical event. For example, a patient event may include a diagnosis of the patient of a certain condition, an incident or episode involving the patient, a symptom experienced by the patient, a test or diagnostic procedure performed on the patient, beginning or ending of a treatment regime for the patient, a change to one or more conditions of the patient (e.g., progression of a disease to a later stage, remission, etc.), a lack of occurrence of particular patient events, a period of time elapsing without events occurring, or any other form of occurrence associated with a patient.

While medical data is provided by way of example, patient data may include a wide variety of data from other sources. For example, patient data may include social media posts or other social media data associated with the patient, shopping or purchasing records associated with the patient (e.g., credit card data, purchase receipts, etc.), fitness or health data (e.g., data from a wearable device, etc.), biometric data, browsing history of the patient, media consumption trends (e.g., movie or show streaming history, podcast listening history, etc.), location information (e.g., geotagged photographs, GPS data, etc.), environmental conditions, genomic data, imaging data like radiological and pathology data, biometric data, medical record data, demographic data, healthcare claims data, financial data, family member history, household information data, living information data, educational data, employment data, or a wide variety of other forms of data associated with a patient. Such patient data may be accessed from data sources 130, as described in further detail below.

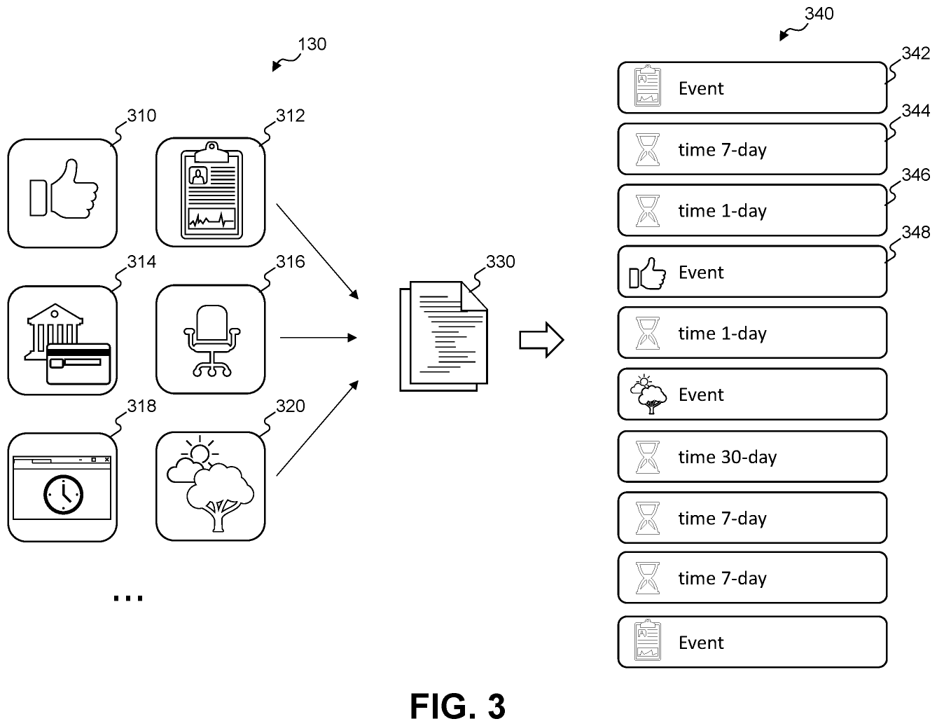

FIG. 3 is a block diagram showing example data sources 130 from which patient event data may be derived, consistent with the disclosed embodiments. As described above, patient data sources 130 may include a wide variety of sources from which patient data may be accessed. For example, data sources 130 may include one or more of data sources 310, 312, 314, 316, 318, or 320. Patient data 330 may be accessed from one or more of these data sources.

In some embodiments, data sources 130 may include social media data 310. Social media data may include data from any form of website or application that enables users to create and share content or to network with others. This may include websites or applications such as Instagram™, Twitter™, Facebook™ Pinterest™, LinkedIn™, TikTok™, YouTube™, Reddit™, or the like. In some embodiments, social media data 310 may include data from dating websites, video game platforms, music streaming services, shopping services, or any other websites or applications that may have social media aspects (e.g., discussion boards, comment sections, sharing features, messaging capabilities, etc.). Social media data 310 may include any data accessible from or stored in association with these services. Example data may include URLs of social media apps or sites visited, posts by the individual, posts viewed by the individual, followers of the individual, accounts followed by the individual, location information, time spent on various social media apps and sites, demographic information, interests and sentiments, or any other activity or statistics associated with a social media platform.

As another example, data sources 130 may include medical data 310. Medical data 310 may include any data associated with the diagnosis, treatment, or other aspects of caring for a patient. In some embodiments, medical data 310 may be included in one or more electronic medical records (EMRs). Medical data 310 may include a variety of patient information, such as a patient name, a patient age, a patient gender, a medical identification number, a physician name, a medical center name, a visit date, a visit result, a test, a test result, a diagnosis, a prognosis, a medication, a dosage, a disease, a medical condition, and/or any other information relevant to patient health. In some embodiments, medical data 310 may be stored as structured information, organized into a predetermined data structure. Alternatively or additionally, medical data 310 may be stored as unstructured information not stored in a particular structure. For example, unstructured data may include handwritten text, pdf documents, text documents, images, or the like. In some embodiments, medical data 310 may be stored in or converted to a standardized format. For example, medical data 310 may include data represented in HL7's Fast Healthcare Interoperability Resources (FHIR) standard. One skilled in the art would recognize various other suitable formats, standards, and protocols may also be used.

As another example, data sources 130 may include financial data 314. For example, financial data 314 may include invoices, receipts, transactions (e.g., credit card transactions, checks, mobile payment transactions, etc.), or any other data that may reflect purchases, redemptions, savings, or spending by the patient. In some embodiments, data sources 130 may include employment data 316. Employment data 316 may include any information associated with the employment or career progression of a patient. For example, this may include current or past positions held by the patient, job titles, hiring events, firing or termination events, promotions, information about coworkers or colleagues, or the like. In some embodiments, data sources 130 may include browsing history data 318. Browsing history data 318 may include any data recorded in association with access to webpages, web applications, or other internet-based sources. For example, browsing history data 318 may include a search history (including individual search terms, search results, etc.), a navigation history, a navigation frequency, or the like. Data sources 130 may further include environmental data 320. Environmental data 320 may include any data associated with environmental conditions of the patient. For example, this may include air quality data, temperatures, sun exposure (e.g., UV indices, etc.), weather conditions, humidity, pressure, location data, infectious disease data, water quality data, or the like.

Patient data 330 may include any combination of the various data described herein. For example, computing device 110 and/or server 120 may compile data from two or more of these various data sources. While various examples are provided herein, the examples are provided for purpose of illustration, and the present disclosure is not limited to any particular examples or combinations of the examples provided.

From patient data 330, various patient events 340 may be extracted. As shown in FIG. 3, patient events 340 may include events compiled from multiple data sources. For example, patient events 340 may include a medical-related event 342, such as a date the patient was diagnosed with diabetes. Patient events 340 may include a medical-related event 348, such as a photo of the patient at an ice cream parlor. Patient events 340 may be extracted from patient data 330 in various way. For example, this may include various optical character recognition (OCR) techniques, image recognition algorithms, trained machine learning models, or the like.

In some embodiments, various features may be extracted from the patient data and mapped to a common vocabulary (e.g., stored on server 120 or computing device 110). For example, this may include extracting features such as medical codes, medications, text from codes and doctor notes, age data, gender data, provider identifiers, start and end dates of services or conditions, or the like. Using the example from FIG. 3, event 342 may be represented in a tokenized format, such as “diag_diabetes,” or various other suitable formats. Similarly, event 348 may be represented as “consume_icecream” or a similar token. In some embodiments, two or more events reflected in patient data may be merged or consolidated. For example, server 120 (or computing device 110) may recognize that two events reflected in data from different sources refer to the same event. Accordingly, these events may be consolidated to avoid duplicate events. These tokens are provided by way of example only, and one of skill in the art would recognize various other formats that may be used.

In some embodiments, patient events 340 may include time information associated with various events. For example, one or more of patient events 340 may include timestamp information indicating a time or date the event occurred. In some embodiments, the time information may be expressed in relative terms. For example, patient events 340 may include one or more timing events, such as events 344 and/or 346, which may indicate the passage of time between events 342 and 346. In this example, event 344 may represent the passing of seven days and event 346 may represent the passing of one day. Accordingly, a total of eight days may have elapsed between events 342 and 346. In some embodiments, these events may be represented as tokens and generated by computing device 110 or server 120. For example, events 344 and 346 may include tokens such as “7 day” and “1 day,” respectively. While the example patient events 340 shown in FIG. 3 span a relatively short timeframe, the patient event data disclosed herein may span longer timeframes, such as several years, several decades, etc.

In some embodiments, the common vocabulary may further be correlated with one or more feature values. For example, events 342, 344, 346, and 348 may be mapped to items 457, 17, 16, and 2232 within a vocabulary, respectively. Accordingly, patient events 340 may be represented as a numerical string: [457, 17, 16, 2231, . . . ]. In some embodiments, patient events 340 may further include other forms of information associated with a patient, such as demographic information. For example, the common vocabulary may include tokens such as: 1. genderfemale; 2. gendermale; . . . 5. age0-1; 6. age1-2; 7. age2-5; 8. age5-10; . . . 18. age55-60; etc. Accordingly, a female patient aged 57 may include vocabulary terms 2 and 18. Thus patient events 340 may be represented as a numerical string: [2, 18, 457, 17, 16, 2231, . . . ]. In some embodiments, the vocabulary may include other terms, such as an identification number of a patient, a provider, or an organization. It is to be understood that the various example tokens and vocabulary items are provided by way of example, and various other formats may be used to represent patient data and/or events.

Once a sequence has been extracted it may be input into the model. As a result, the trained artificial intelligence model may output a sequence of predicted events, which may follow the same or similar feature mapping as the input sequence. As a result, a series of future events may be predicted for the patient. Notably, the resulting sequence may predict a wide variety of patient events associated and may not be limited to any particular prompt or event type. In some embodiments the output sequence may include time tokens (similar to the input sequence described above) to establish relative dates of the various events.

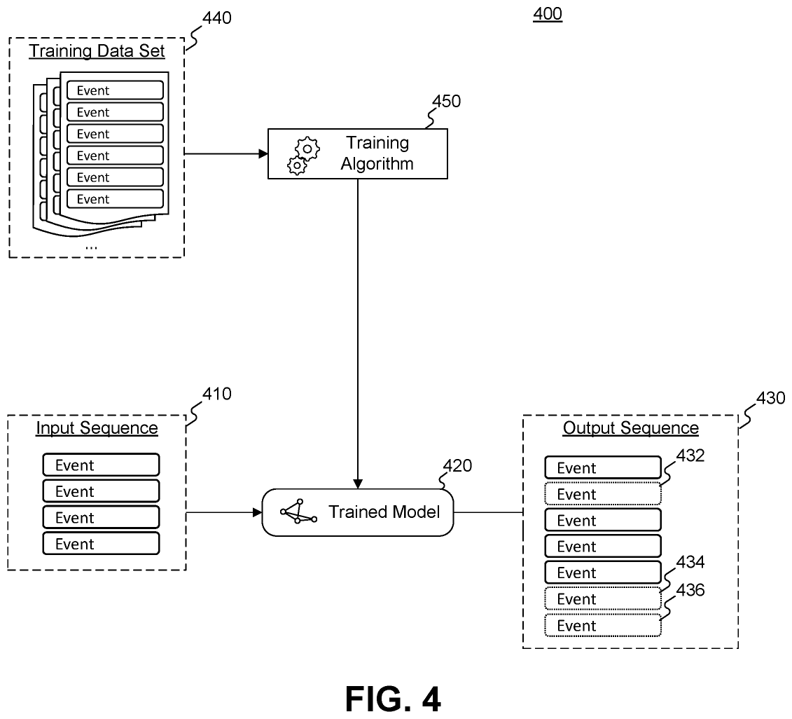

FIG. 4 is a block diagram illustrating an example process 400 for training and applying a machine learning model consistent with the disclosed embodiments. As shown in FIG. 4, an input sequence of events 410 may be input into a trained model 420. As a result, an output sequence 430 may be generated by the trained model. Input sequence 410 may correspond to events extracted from patient data. For example, input sequence 410 may correspond to event data 340, described above.

Trained model 420 may be trained to predict one or more additional events based on input sequence 410. Accordingly, output sequence 430 may include one or more events 432, 434, and 436 not present in input sequence 410. In some embodiments, this may include future events that have not occurred, such as events 434 and 436, which may be associated with dates in the future. Alternatively or additionally, this may include predicting past events that may have occurred but are not represented in input sequence 410. For example, this may include event 432 shown in FIG. 4.

Trained model 420 may include any type of artificial intelligence model capable of predicting a sequence of events. In some embodiments, trained model 420 may be a deep learning model used for natural language processing. For example, the trained artificial intelligence model may be a sequence-to-sequence (Seq2Seq) model or similar type of recurrent neural network. For example, the sequence-to-sequence model may be a Transformer (or “Transformer-XL”) model. While a sequence-to-sequence model is provided by way of example, various other forms of models may be used as an alternative or in addition to the sequence-to-sequence model, including but not limited to a logistic regression, a linear regression, a regression, a random forest, a K-Nearest Neighbor (KNN) model, a K-Means model, a decision tree, a cox proportional hazards regression model, a Naïve Bayes model, a Support Vector Machines (SVM) model, a gradient boosting algorithm, Long-Short Term Memory neural networks (LSTM), convolutional neural networks (CNN), recurrent neural networks (RNN), multi-layer perceptrons (MLP), or any other form of machine learning model or algorithm.

Trained model 420 may be trained in various ways. In some embodiments, trained model 420 may be trained using a training data set 440, which may be fed into a training algorithm 450. As a result of this training process, trained model 420 may be developed. As indicated in FIG. 4, training data set 440 may include one or more sequences of events. For example, training data set 440 may include sequences of events similar to patient events 340 for many different patients. Accordingly, trained model 420 may be trained to generate output sequence 430 based on the most probable sequence of events indicated in training data set 440.

In some embodiments, training data set 440 may include a vast amount of data. For example, training data set 440 may include thousands, millions, or even billions of event sequences for different patients, each including data from a wide variety of different data sources. Accordingly, trained model 420 may be a robust tool capable of detecting trends in patient data that would otherwise be impossible to identify. For example, a doctor or other medical professional typically does not have access to the wide variety of data represented in input sequence 440. Indeed, a physician typically does not have access to environmental conditions experienced by a patient, purchases made by the patient, the patient's social media posts, or websites visited by the patient. Even if they did, a physician is not trained to understand how variations in this data would indicate future events, such as a medical emergency. Trained model 420 may be capable of detecting minute variations or trends in data from many different sources and generating a prediction of a future event that would otherwise be unpredictable. In other words, training data set 440 may reflect complicated relationships between trends in different types of events and how they are correlated to later events that occur. Based on this training data set, trained model 420 may be configured to predict similar outcomes based on input sequence 440.

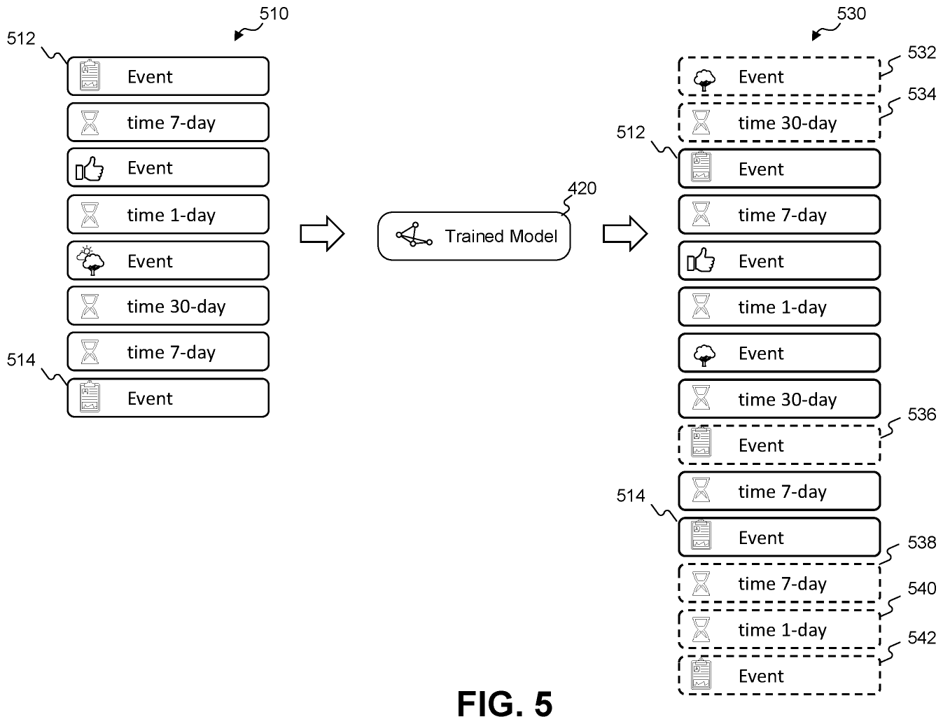

FIG. 5 is a block diagram illustrating an example output sequence 530 that may be generated using trained model 420, consistent with the disclosed embodiments. As described above, input sequence 510 may be input into trained model 420 and used to generate output sequence 530. For example, input sequence 510 and output sequence 530 may correspond to input sequence 410 and output sequence 430, respectively. While represented graphically in FIG. 5, it is to be understood that one or both of input sequence 510 and output sequence 530 may be represented in various other formats. For example, input sequence 510 and output sequence 530 may be represented in a canonical form (e.g., using a list of predefined terms) and may further be mapped to a predefined list of numerical values, as described above.

Output sequence 530 may include one or more predicted events not included in input sequence 510. For example, output sequence 530 may include events 532, 534, 536, 538, 540, and 542, as shown in FIG. 5. In some embodiments, the predicted events may occur after input sequence 510. For example, predicted event 542 may include a medical event predicted to occur at some time in the future. Alternatively or additionally, event 542 may include a predicted or recommended treatment for the patient based on the events included in input sequence 510. For example, trained model 420 may be trained to identify treatments most likely to result in a desired outcome based on training data set 440.

The predicted events are not necessarily limited to future events. For example, event 536 may be an event interspersed in the events of input sequence 510. For example, event 536 may be a Transient Ischemic Attack (TIA) or other episode that may be predicted to have occurred but not diagnosed or otherwise reflected in input sequence 510. As another example, event 532 may be predicted to have occurred prior to the events in input sequence 510. In this example, event 532 may reflect an exposure to a pollutant or other environmental condition that may have contributed to one or more events in output sequence 530.

In some embodiments, the timings of predicted events may also be output by trained model 420. For example, this may be achieved through including predicted time tokens, similar to time tokens 344 and 346, as described above. In this example, output sequence 530 may include time tokens 538 and 540 indicating event 542 is predicted to occur 8 days after event 514. Accordingly, medical professionals or other individuals (i.e., user 112), may be forewarned of event 542 and may take precautionary or preventative measures. Likewise, time token 534 may indicate a timing of event 532 relative to event 512.

According to some embodiments, not all of the input sequence of events may be extracted from patient data. In some embodiments, one or more of the events may be hypothetical events. For example, event 514 may represent a treatment contemplated for a particular patient but that has not yet been implemented. Accordingly, events extracted from patient data may be supplemented with this hypothetical treatment event. Predicted event 542 may thus represent a predicted outcome of the treatment. For example, event 542 may represent a predicted adverse reaction to a treatment, a cure of a condition, a progression stage of a disease, or the like. Accordingly, trained model 420 may be useful in evaluating the downstream effects of particular events that have not yet occurred.

In some embodiments, trained model 420 may further be configured to determine probabilities of predicted events. In other words, trained model 420 may be used to generate a value indicating a degree of likelihood that a predicted event will occur. In some embodiments, the probability may be output directly by trained model 420, for example, as part of output sequence 430. Alternatively or additionally, the probability may be calculated based on the output of trained model 420. One example technique for determining this probability is to run trained model 420 multiple times and perform a statistical analysis of the results. The relative frequency at which an event is predicted to occur may reflect a probability of occurrence of the event. This statistical approach may also indicate probabilities of when an event will occur, whether a certain event will occur before or after another predicted event, or the like.

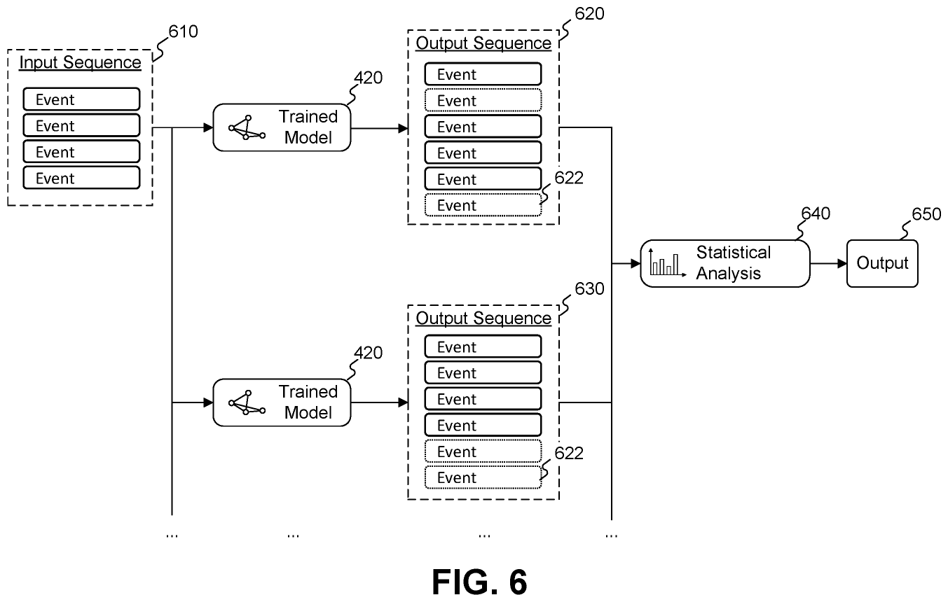

FIG. 6 is a block diagram illustrating an example statistical analysis 640 that may be performed, consistent with the disclosed embodiments. For example, an input sequence 610 may be input into trained model 420, similar to input sequence 410, as described above. This may result in an output sequence 620, as shown. Input sequence 610 may be input to trained model 420 a second time to generate output 630. These outputs may be compared to determine a more robust prediction of future events, as represented by output 650.

For example, this may include performing statistical analysis 640 on output sequences 620 and 630. Statistical analysis 640 may include a wide variety of different statistical algorithms or techniques, as would be recognized by one of skill in the art. For example, statistical analysis 640 may include a Monte Carlo simulation used to predict a range of future outcomes. Accordingly, based on statistical analysis 640 system 100 may be used to evaluate the likelihood of different future outcomes. In embodiments, where hypothetical events are added to input sequence 610, statistical analysis 640 may be used to evaluate the probabilities of different results of the hypothetical event. Different hypothetical events may also be compared to assess their relative efficacies.

In some embodiments, statistical analysis 640 may be used to predict a likelihood an event will occur in a predicted timeframe. For example, both of output sequences 620 and 630 may include the same event 622. However, event 622 may be predicted to occur at different timeframes in each of sequences 620 and 630. Accordingly, statistical analysis 640 may reflect a range of times in which event 622 may occur, a probability distribution of times for event 622, a most likely date for event 622, or the like. While statistical analysis 640 is illustrated in FIG. 6 to be based on two output sequences for purposes of simplicity, one of skill in the art would recognize that any number of output sequences may be used in statistical analysis 640.

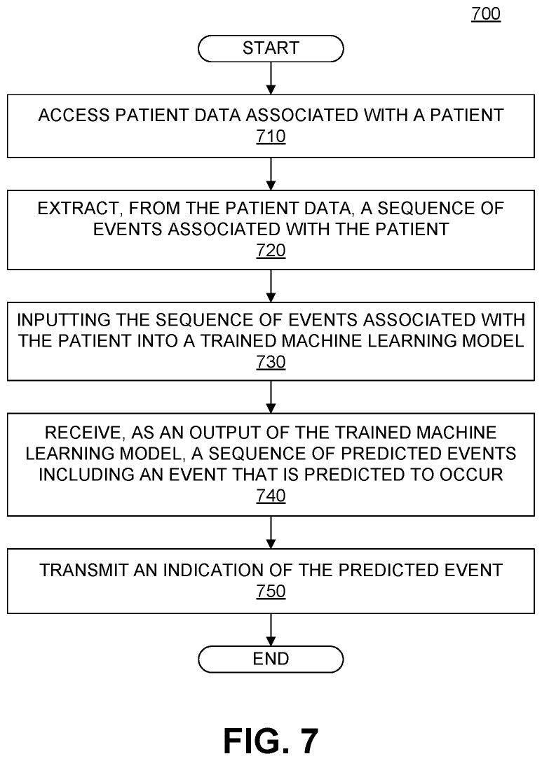

FIG. 7 is a flowchart showing an example process 700 for providing medical data to predict patient events, consistent with the disclosed embodiments. Process 700 may be performed by at least one processor of a computing device, such as processor 210, as described above. Alternatively or additionally, some or all of process 700 may be performed by at least one processor of a server, such as process 230. It is to be understood that throughout the present disclosure, the term “processor” is used as a shorthand for “at least one processor.” In other words, a processor may include one or more structures that perform logic operations whether such structures are collocated, connected, or dispersed. In some embodiments, a non-transitory computer readable medium may contain instructions that when executed by a processor cause the processor to perform process 700. Further, process 700 is not necessarily limited to the steps shown in FIG. 7, and any steps or processes of the various embodiments described throughout the present disclosure may also be included in process 700, including those described above with respect to, for example, FIGS. 3, 4, 5, and 6.

In step 710, process 700 may include accessing, from one or more data sources, patient data associated with a patient. For example, this may include accessing patient data 330 from data sources 130, as described above. The patient data may come from a wide variety of sources and may include many different types of patient data. For example, as described above, the patient data may include social media data, purchasing records, fitness data, biometric data, medical record data, demographic data, healthcare claims data, financial data, family member history, household information data, living information data, educational data, employment data, browsing history data, media consumption data, location history information, environmental condition data, video data, genomic data, imaging data, or any other types of data associated with a patient. According to some embodiments, the patient data may include at least one patient medical record including data represented in a canonical form. For example, the canonical form may include FHIR or similar standards. In some embodiments, step 710 may further include converting a medical record or other data into the canonical form. For example, step 710 may include accessing at least one patient medical record stored in a noncanonical form; and extracting information to generate the data represented in the canonical form.

In step 720, process 700 may include extracting, from the patient data, a sequence of events associated with the patient. For example, this may include extracting patient events 340, as described above. As described above, the plurality of features may include at least one time token representing a time interval between at least two of the plurality of historical events. For example, this may include time tokens 344 or 346 described above. In some embodiments, step 720 may include consolidating two or more events. For example, mapping the plurality of historical events to the plurality of features includes mapping at least two of the plurality of historical events into a composite feature.

In some embodiments, the sequence of events may be represented using a predetermined vocabulary, as described above. Accordingly, extracting the sequence of events associated with the patient may include identifying a plurality of historical events represented in the patient data, and mapping the plurality of historical events to a plurality of features represented using a predetermined vocabulary. In some embodiments, the predetermined vocabulary may include additional data associated with a patient. For example, the predetermined vocabulary may include an identification number of at least one of an individual patient, a provider, or an organization.

According to some embodiments, the sequence of events may include hypothetical events that have not yet occurred. Accordingly, step 720 may include generating at least one hypothetical event not represented in the patient data. and mapping the at least one hypothetical event to the plurality of features represented using the predetermined vocabulary. For example, the at least one hypothetical event may include a potential treatment for the patient.

In step 730, process 700 may include inputting the sequence of events associated with the patient into a trained machine learning model. For example, this may include inputting input sequence of events 410 into trained model 420. As described above, the trained model may include a variety of different types of models, such as artificial intelligence models. In some embodiments, the trained machine learning model may include a sequence-to-sequence model, such as a Transformer model. According to some embodiments, the trained machine learning model may include internal network weights. In some embodiments, at least one of the internal network weights may be used for an independent use case. For example, the independent use case may include creating embeddings, creating probabilities, or creating medical concept relationships. In some embodiments, the independent use case may include producing an embedding to define a token or sequence of events. The embedding may thus enable finding at least one of: a similarity among two or more sequences or a similarity between the embedding and at least one other token embedding.

In step 740, process 700 may include receiving, as an output of the trained machine learning model, a sequence of predicted events associated with the patient. For example, this may include receiving output sequence 430 (or sequences 530, 620, or 630), as described above. The sequence of predicted events may include at least one event that is predicted to occur (or have occurred). For example, sequence of predicted events may include events 532, 534, 536, 538, 540, and 542, as shown in FIG. 5. In some embodiments, the predicted events may include events predicted to have occurred already. Accordingly, the predicted events may include at least one event predicted to have occurred before the sequence of events and/or at least one event interspersed with the sequence of events. As described above, the input sequence of events may be represented using a predetermined vocabulary. In some embodiments, the sequence of predicted events may also include a plurality of predicted events represented using the predetermined vocabulary. According to some embodiments, the plurality of predicted events may include at least one time token representing a time interval between at least two of the plurality of predicted events, as described above.

In some embodiments, the sequence of events may be represented as a probability distribution. For example, the at least one predicted event may be represented as a probability distribution of possible next events or a probability distribution of possible previous events. In some embodiments, the predicted events may not be associated with a particular patient. For example, the patient may be a hypothetical patient and the sequence of predicted events may include a sequence of events predicted to occur for the hypothetical patient or a population of patients.

In step 750, process 700 may include transmitting an indication of the at least one predicted event. For example, this may include transmitting an alert to user 112, which may be the patient, a doctor, a nurse, a relative, an organization, or any other entity associated with the patient. In some embodiments, the indication may identify the predicted event, a likelihood of occurrence, a timeframe of the predicted event, or the like.

In some embodiments, process 700 may further include determining a probability of occurrence of the at least one predicted event. For example, this may include using statistical analysis 640 as described above. Accordingly, process 700 may include receiving, as an output of the trained machine learning model, at least one additional sequence of predicted events associated with the patient (e.g., output sequence 630) and determining the probability of occurrence of the at least one predicted event based on a statistical analysis of the sequence of predicted events and the at least one additional sequence of predicted events. For example, the statistical analysis may include a Monte-Carlo simulation. In some embodiments, determining the probability of occurrence of the at least one predicted event includes determining a probability that the at least one predicted event will occur within a specified timeframe. Alternatively or additionally, determining the probability of occurrence of the at least one predicted event may include determining a probability distribution of the sequence of events. Transmitting the indication of the at least one predicted event in step 750 may include generating an alert for an entity associated with treatment of the patient, the alert indicating the probability of occurrence of the at least one predicted event.

As explained above, the input sequence of events may include at least one hypothetical event not represented in the patient data. For example, the at least one hypothetical event may include a potential treatment for the patient. In some embodiments, process 700 may be used to evaluate multiple potential treatments. For example, process 700 may further include receiving, as an output of the trained machine learning model, an alternate sequence of predicted events associated with the patient. The alternate sequence of predicted events may be determined based on at least one alternate potential treatment for the patient, where the at least one alternate potential treatment is different from the potential treatment. Process 700 may further include comparing the sequence of predicted events associated with the patient with the alternate sequence of predicted events associated with the patient and generating a recommendation for at least one of the potential treatments or the alternate potential treatment based on the comparison.

It is to be understood that the disclosed embodiments are not necessarily limited in their application to the details of construction and the arrangement of the components and/or methods set forth in the following description and/or illustrated in the drawings and/or the examples. The disclosed embodiments are capable of variations, or of being practiced or carried out in various ways.

The disclosed embodiments may be implemented in a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowcharts or block diagrams may represent a software program, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant virtualization platforms, virtualization platform environments, trusted cloud platform resources, cloud-based assets, protocols, communication networks, security tokens and authentication credentials, and code types will be developed, and the scope of these terms is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A computer-implemented method for using an expert system for providing medical data to predict patient events, the method comprising:
accessing, from one or more data sources, patient data associated with a patient;
extracting, from the patient data, a sequence of events associated with the patient;
inputting the sequence of events associated with the patient into a trained machine learning model;
receiving, as an output of the trained machine learning model, a sequence of predicted events associated with the patient, the sequence of predicted events including at least one event that is predicted to occur; and
transmitting an indication of the at least one predicted event.

2. The computer-implemented method of claim 1, wherein the trained machine learning model includes a sequence-to-sequence model.

3. The computer-implemented method of claim 2, wherein the sequence-to-sequence model includes a Transformer model.

4. The computer-implemented method of claim 1, wherein the patient data includes at least one of social media data, purchasing records, fitness data, biometric data, medical record data, demographic data, healthcare claims data, financial data, family member history, household information data, living information data, educational data, employment data, browsing history data, media consumption data, location history information, environmental condition data, video data, genomic data, or imaging data.

5. The computer-implemented method of claim 1, wherein the patient data includes at least one patient medical record including data represented in a canonical form.

6. The computer-implemented method of claim 5, wherein accessing the patient data associated with the patient includes:
accessing at least one patient medical record stored in a noncanonical form; and
extracting information to generate the data represented in the canonical form.

7. The computer-implemented method of claim 1, wherein extracting the sequence of events associated with the patient includes:
identifying a plurality of historical events represented in the patient data; and
mapping the plurality of historical events to a plurality of features represented using a predetermined vocabulary.

8. The computer-implemented method of claim 7, wherein the plurality of features includes at least one time token representing a time interval between at least two of the plurality of historical events.

9. The computer-implemented method of claim 7, wherein mapping the plurality of historical events to the plurality of features includes mapping at least two of the plurality of historical events into a composite feature.

10. The computer-implemented method of claim 7, wherein extracting the sequence of events associated with the patient further includes:
generating at least one hypothetical event not represented in the patient data; and
mapping the at least one hypothetical event to the plurality of features represented using the predetermined vocabulary.

11. The computer-implemented method of claim 10, wherein the at least one hypothetical event includes a potential treatment for the patient.

12. The computer-implemented method of claim 11, wherein the operations further include:
receiving, as an output of the trained machine learning model, an alternate sequence of predicted events associated with the patient, the alternate sequence of predicted events associated with the patient being determined based on at least one alternate potential treatment for the patient, the at least one alternate potential treatment being different from the potential treatment;
comparing the sequence of predicted events associated with the patient with the alternate sequence of predicted events associated with the patient; and
generating a recommendation for at least one of the potential treatments or the alternate potential treatment based on the comparison.

13. The computer-implemented method of claim 7, wherein the sequence of predicted events includes a plurality of predicted events represented using the predetermined vocabulary.

14. The computer-implemented method of claim 13, wherein the plurality of predicted events includes at least one time token representing a time interval between at least two of the plurality of predicted events.

15. The computer-implemented method of claim 7, wherein the predetermined vocabulary includes an identification number of at least one of an individual patient, a provider, or an organization.

16. The computer-implemented method of claim 1, wherein the method further includes determining a probability of occurrence of the at least one predicted event.

17. The computer-implemented method of claim 16, wherein determining the probability of occurrence of the at least one predicted event includes:
receiving, as an output of the trained machine learning model, at least one additional sequence of predicted events associated with the patient; and
determining the probability of occurrence of the at least one predicted event based on a statistical analysis of the sequence of predicted events and the at least one additional sequence of predicted events.

18. The computer-implemented method of claim 17, where the statistical analysis includes a Monte-Carlo simulation.

19. The computer-implemented method of claim 16, wherein determining the probability of occurrence of the at least one predicted event includes determining a probability of the at least one predicted event will occur within a specified timeframe.

20. The computer-implemented method of claim 16, wherein transmitting the indication of the at least one predicted event includes generating an alert for an entity associated with treatment of the patient, the alert indicating the probability of occurrence of the at least one predicted event.

21. The computer-implemented method of claim 16, wherein determining the probability of occurrence of the at least one predicted event includes determining a probability distribution of the sequence of events.

22. The computer-implemented method of claim 1, wherein the trained machine learning model includes internal network weights, wherein at least one of the internal network weights may be used for an independent use case, the independent use case including at least one of: creating embeddings, creating probabilities, or creating medical concept relationships.

23. The computer-implemented method of claim 19, wherein the independent use case includes producing an embedding to define a token or sequence of events.

24. The computer-implemented method of claim 23, wherein the embedding enables finding at least one of: a similarity among two or more sequences or a similarity between the embedding and at least one other token embedding.

25. The computer-implemented method of claim 1, wherein the trained machine learning model includes an artificial intelligence model.

26. The computer-implemented method of claim 1, wherein the method further includes receiving, as an output of the trained machine learning model, at least one of: an event predicted to have occurred before the sequence of events or an event interspersed with the sequence of events.

27. The computer-implemented method of claim 1, wherein the at least one predicted event is represented as at least one of: a probability distribution of possible next events or a probability distribution of possible previous events.

28. The computer-implemented method of claim 1, wherein the patient is a hypothetical patient and the sequence of predicted events includes a sequence of events predicted to occur for the hypothetical patient or a population of patients

29. A non-transitory computer readable medium including instructions that, when executed by at least one processor, cause the at least one processor to perform operations for using an expert system for providing medical data to predict patient events, the operations comprising:
accessing, from one or more data sources, patient data associated with a patient;
extracting, from the patient data, a sequence of events associated with the patient;
inputting the sequence of events associated with the patient into a trained machine learning model;
receiving, as an output of the trained machine learning model, a sequence of predicted events associated with the patient, the sequence of predicted events including at least one event that is predicted to occur; and
transmitting an indication of the at least one predicted event.

30. A system for using an expert system for providing medical data to predict patient events, the system comprising:
at least one processor comprising circuitry and a memory, the memory including instructions that when executed by the circuitry cause the at least one processor to:
access, from one or more data sources, patient data associated with a patient;
extract, from the patient data, a sequence of events associated with the patient;
input the sequence of events associated with the patient into a trained machine learning model;
receive, as an output of the trained machine learning model, a sequence of predicted events associated with the patient,
the sequence of predicted events including at least one event that is predicted to occur; and
transmit an indication of the at least one predicted event.
